(19) United States
(12) Patent Application Publication
(10) Pub. No.: US 2023/0041271 A1
(43) Pub. Date: Feb. 9, 2023

(54) Mitochondria-Targeted Polypeptide, Preparation Method thereof, and Use thereof

(71) Applicants: Shenzhen Campus of Sun Yat-sen University, Shenzhen (CN); SUN YAT-SEN UNIVERSITY, Guangzhou (CN)

(72) Inventors: Qicai Xiao, Guangzhou (CN); Liqian Gao, Guangzhou (CN); Shizhe Zhou, Guangzhou (CN)

(21) Appl. No.: 17/828,609

(22) Filed: May 31, 2022

(30) Foreign Application Priority Data
Jul. 26, 2021 (CN) 2021108461006

(51) Int. Cl. C07K 7/06; A61K 47/54; A61K 47/62; A61K 49/00; A61K 31/704; A61K 31/4745

(52) U.S. Cl. CPC C07K 7/06; A61K 47/545; A61K 47/549; A61K 47/62; A61K 49/0056; A61K 31/704; A61K 31/4745

(57) ABSTRACT

Disclosed are a type of mitochondria-targeted polypeptides, the preparation method and the uses thereof. The polypeptide is abbreviated as MTP. The synthesis method of the present disclosure is simple, and the mitochondria-targeted polypeptide prepared by the method can specifically target the mitochondria of cells and are basically non-toxic to cells. In addition, these synthesized polypeptides demonstrate good cell-membrane-penetrating properties, and can conveniently undergo further multi-functional derivation and modification, thereby providing a potential delivery tool for the preparation of a mitochondria-targeted medicament.

### CROSS-REFERENCE TO RELATED APPLICATION

The present application claims the benefit of and priority to Chinese Patent Application No. 2021108461006, filed on Jul. 26, 2021, the entire disclosures of which are incorporated herein by reference.

### TECHNICAL FIELD

The present disclosure belongs to the field of biomedicine, and particularly involves a type of mitochondria-targeted polypeptides, the preparation method and uses thereof.

### BACKGROUND

Cancer (malignant tumor) has become a major disease that seriously endangers human health and has brought enormous pressure to people's lives and economic and social development. Although great progress has been made in the prevention, detection and treatment of cancer at present, there is currently still no effective therapy for treating tumors. A long-standing problem in cancer chemotherapy is the non-specific distribution of therapeutic drugs and the lack of tumor selectivity, which causes systemic toxicity and other serious side effects during treatment, such as hair loss, anemia, and kidney, liver and bone marrow damage. Therefore, it is of great significance to develop effective anti-cancer drug delivery systems that can distinguish cancer cells from normal cells, thereby improving the efficacy of anti-tumor therapies.

Mitochondria, a type of important subcellular organelle in mammalian cells, are energy providers for life activities in cells and called “energy factory” for cells. Mitochondria are involved in many cellular functional activities including cell cycle, cell metabolism, apoptosis, signal transduction, etc. Dysfunction of mitochondria is closely related to lot of diseases such as cancer, obesity, diabetes, cardiovascular diseases, and neurodegenerative diseases. Therefore, mitochondria are an important type of targets for the treatment of diseases and have attracted more and more attention from researchers in recent years. Compared with normal cells, tumor cells grow and proliferate faster and require more energy. In order to meet the needs of rapid cell growth, tumor cells often contain more mitochondria. Targeting mitochondria interferes with the energy supply to cells, which disrupts the biological functions of the cells. However, the entry of an exogenous substance into mitochondria requires penetration of the cell membrane and a complex mitochondrial membrane region composed of an outer mitochondrial membrane, an inner mitochondrial membrane and an intermembrane cavity. Therefore, targeted delivery of an exogenous active substance to mitochondria is a very challenging task, and the study of mitochondria-targeted delivery carriers has an important scientific research significance and clinical value.

In order to achieve the selective delivery of an exogenous active substance to mitochondria and specific accumulation therein, a variety of mitochondria-targeted delivery systems have been studied and reported to date. In the related art, lipophilicity-based cations such as triphenylphosphine (TPP) have been successfully applied to mitochondria-targeted delivery of various small-molecule compounds; and liposome-, polymer-, and hydrogel-based nanoparticles and biodegradable nanoparticles have been reported for mitochondria-targeted delivery of macromolecules such as proteins. In addition, due to the fact that polypeptide compounds have excellent biocompatibility, the polypeptide compounds can be synthesized simply and conveniently, and are easy to be multi-functionally derived and modified; mitochondria-targeted delivery systems based on polypeptides have attracted more and more attention. Cell-penetrating peptides (CPPs) are polypeptides composed of 4 to 30 amino acids with one to several positive charges, which can electrostatically interact with negatively charged cell membranes to facilitate cellular uptake. Currently, various sources of cell-penetrating peptides have been developed for the delivery of small molecules, proteins and nucleic acids. However, it is difficult to modify conventional mitochondria-targeted peptides with diversification and multi-functionality, and there have been currently no reported peptide-based mitochondria-targeted delivery systems that can simultaneously perform tumor-targeted delivery and traceless mitochondria-targeted release. Therefore, the development of a structurally simple and novel mitochondria-targeted peptide, which can selectively deliver an exogenous active substance to the mitochondria of tumor cells and can release the exogenous active substance in response to the tumor cell microenvironment in a traceless manner, thereby disrupting mitochondrial and cellular functions, is of great significance for the treatment of mitochondria-related diseases such as tumors.

### SUMMARY

The present disclosure aims to solve at least one of the above-mentioned technical problems existing in the prior art. Provided is a mitochondria-targeted polypeptide, which solves the technical problems that existing mitochondria-targeted peptides are difficult to be multi-functionally targeted modified and the carried active substance cannot be traceless released in mitochondria.

The present disclosure further provides a method for preparing the above-mentioned polypeptide.

The present disclosure further provides the use of the above-mentioned polypeptide.

According to one aspect of the present disclosure, provided is a mitochondria-targeted polypeptide, wherein the polypeptide is abbreviated as MTPs, and the general structural formula of the polypeptide is as shown below in Formula I:

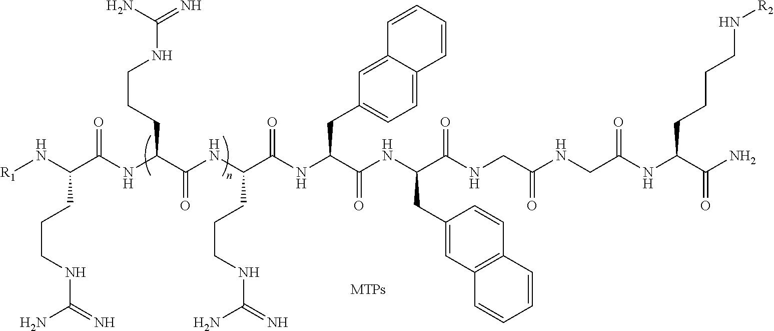

wherein n≥0, R1 is an amino protecting group or a tumor targeting ligand, and R2 is at least one selected from the group consisting of hydrogen, fluorescent groups, and drug groups.

In some embodiments of the present disclosure, the amino protecting group is at least one selected from the group consisting of acetyl, propionyl and butyryl.

In some embodiments of the present disclosure, the tumor targeting ligand is at least one selected from the group consisting of folic acid, nucleic acid aptamers, RGD-targeting peptides, and biotin.

In some embodiments of the present disclosure, the fluorescent group is at least one selected from the group consisting of rhodamine fluorophore and derivatives thereof, fluorescein isothiocyanate and derivatives thereof, or pyrene-based fluorophore and derivatives thereof.

In some embodiments of the present disclosure, the drug group is at least one selected from the group consisting of doxorubicin, camptothecin and derivatives thereof.

In a second aspect of the present disclosure, provided is a method for preparing the above-mentioned polypeptide, the method comprising the following steps: preparing a polypeptide chain by an Fmoc solid-phase synthesis process, and cleaving and purifying the polypeptide chain to obtain the polypeptide.

In some embodiments of the present disclosure, the method for preparing the polypeptide comprises the following steps:

S1. swelling a resin, washing and deprotecting the resin, and then condensing a first Fmoc-amino acid with the resin under the catalysis of a polypeptide condensing agent; after the reaction is completed, carrying out deprotection and washing, and then carrying out a Kaiser test to confirm that the deprotection is completed; and then condensing a second amino acid, and repeating the above step until the polypeptide chain synthesis is completed; and

S2. cleaving the synthesized polypeptide chain with a cleavage cocktail; and after solid-liquid separation, adding cold diethyl ether to the liquid phase for precipitation to obtain a crude peptide, and further purifying the crude peptide by liquid chromatography.

In some embodiments of the present disclosure, the resin is Rink Amide resin.

In some embodiments of the present disclosure, the polypeptide condensing agent is HATU.

In some embodiments of the present disclosure, the deprotection involves washing the resin with a 10-30% piperidine/DMF (v/v) solution to remove the Fmoc protecting group.

In some embodiments of the present disclosure, the Kaiser test is a ninhydrin test.

In some embodiments of the present disclosure, an acidic shearing reagent is used for cleaving, wherein the acidic shearing reagent comprises 90-95% of TFA, 2-3% of water, 2-3% of TIPS, and 2-3% of 1,3-dimethoxybenzene.

According to a third aspect of the present disclosure, provided is the use of the above-mentioned polypeptide, wherein the use is for the preparation of a mitochondria-targeted medicament.

In some embodiments of the present disclosure, the medicament comprises a mitochondria-targeted prodrug responsive to endogenous GSH in cells.

In some embodiments of the present disclosure, the use of the polypeptide is for the preparation of a cell-membrane-penetrating peptide.

In some embodiments of the present disclosure, provided is the use of the polypeptide as a drug carrier.

Provided is a pharmaceutical composition comprising the above-mentioned polypeptide.

In some embodiments of the present disclosure, the pharmaceutical composition is in the form of tablets, injection, powder, elixir, capsules, suspension, syrup, pills, or sheet.

The polypeptide prepared according to the embodiment of the present disclosure has at least the following beneficial effects: the mitochondria-targeted polypeptide prepared by the present disclosure is simply synthesized, can specifically target cell mitochondria, can be multi-functionally modified conveniently, has a good cell membrane permeability and can be highly selectively enriched in mitochondria. The co-localization coefficient R of the best mitochondria-targeted polypeptide with a commercial mitochondrial fluorescent localization probe (MitoTracker® Deep Red FM, a near-infrared mitochondrial probe) is as high as 0.84; furthermore, the mitochondrial targeting properties of this mitochondria-targeted polypeptide are substantially unaffected by the delivered fluorescent group; in addition, the mitochondria-targeted polypeptide prepared by the present disclosure has a good biocompatibility and remains basically non-toxic to cells at a concentration of 50 μM. After the polypeptide prepared by the present disclosure is used as a carrier and modified with the tumor targeting ligand biotin and the anti-tumor active drug doxorubicin (Dox), the obtained mitochondria-targeted prodrug can be selectively enriched in mitochondria and can in-situ release the active drug in the mitochondria. In an in vitro cell activity test, the prepared mitochondria-targeted prodrug can selectively kill tumor cells and is basically non-toxic to normal cells. The polypeptide prepared by the present disclosure can deliver active substances with various structures to mitochondria in a targeted manner and provides a potential delivery tool for the preparation of a mitochondria-targeted drug.

### BRIEF DESCRIPTION OF DRAWINGS

The present disclosure will be further illustrated below in conjunction with the accompanying drawings and examples, wherein

FIG. 1 is a graph showing the results of LC-MS characterization of the polypeptide MTP2 in Example 2;

FIG. 2 is a graph showing the results of LC-MS characterization of the polypeptide MTP3 in Example 2;

FIG. 3 is a graph showing the results of LC-MS characterization of the polypeptide MTP4 in Example 2;

FIG. 4 is a graph showing the results of LC-MS characterization of the polypeptide MTP5 in Example 2;

FIG. 5 is a graph showing the results of LC-MS characterization of mitochondria-targeted fluorescent probe 8 obtained after connecting the polypeptide MTP2 to a pyrenyl fluorophore in Example 3;

FIG. 6 is a graph showing the results of LC-MS characterization of mitochondria-targeted fluorescent probe 9 obtained after connecting the polypeptide MTP3 to a pyrenyl fluorophore in Example 3;

FIG. 7 is a graph showing the results of LC-MS characterization of mitochondria-targeted fluorescent probe 10 obtained after connecting the polypeptide MTP4 to a pyrenyl fluorophore in Example 3;

FIG. 8 is a graph showing the results of LC-MS characterization of mitochondria-targeted fluorescent probe 11 obtained after connecting the polypeptide MTPS to a pyrenyl fluorophore in Example 3;

FIG. 9 is a graph showing the results of LC-MS characterization of mitochondria-targeted fluorescent probe 12 obtained after connecting an intermediate pentapeptide to a pyrenyl fluorophore in Example 3;

FIG. 10 is a graph showing the results of LC-MS characterization of the fluorescent probe MTP3-TMR obtained by coupling the polypeptide MTP3 to TMR fluorophore in Example 3;

FIG. 11 is a graph showing the results of LC-MS characterization of compound MTP3-FAM fluorescent probe obtained by coupling the polypeptide MTP3 to FAM in Example 3;

FIG. 12 is mitochondrial co-localization imaging photos of mitochondria-targeted fluorescent probes 8-12 in an experimental example of the present disclosure;

FIG. 13 is mitochondrial co-localization imaging photos of mitochondria-targeted fluorescent probes 13 and 14 in an experimental example of the present disclosure;

FIG. 14 is a graph showing the cell uptake results of mitochondria-targeted fluorescent probes 8-12 in an experimental example of the present disclosure;

FIG. 15 is a graph showing the results of LC-MS characterization of compound 16 in an experimental example of the present disclosure;

FIG. 16 is a graph showing the results of ¹HNMR characterization of compound 19 in an experimental example of the present disclosure;

FIG. 17 is a graph showing the results of ¹H NMR and ¹³C NMR characterization of compound 20 in an experimental example of the present disclosure;

FIG. 18 is an LC-MS graph of mitochondria-targeted prodrug 17 (Bio-MTP3-SS-Dox) in an experimental example of the present disclosure;

FIG. 19 is a schematic diagram showing the GSH-responsive release mechanism of mitochondria-targeted prodrug 17 in a PBS solution in vitro in an experimental example of the present disclosure;

FIG. 20 is a graph showing the GSH-responsive release results of mitochondria-targeted prodrug 17, as detected by HPLC, in an experimental example of the present disclosure, wherein A is a graph of HPLC detection, B is a graph showing the HPLC detection results of mitochondria-targeted compound 16; C is a graph showing the HPLC detection results of mitochondria-targeted prodrug 17; D is a graph showing the release results of mitochondria-targeted prodrug 17 after being placed in a PBS solution at 37° C. for 7 days; and E is a graph showing the results of HPLC detection after co-incubation of mitochondria-targeted prodrug 17 with GSH for 6 h;

FIG. 21 is a graph showing the release results of mitochondria-targeted prodrug 17, as detected by ESI-MS, in an experimental example of the present disclosure;

FIG. 22 is a graph showing the GSH concentration detection results for mitochondria-targeted prodrug 17 in an experimental example of the present disclosure;

FIG. 23 is a graph showing the responsive release results of mitochondria-targeted prodrug 17 in a PBS solution in vitro over time in an experimental example of the present disclosure;

FIG. 24 is a graph showing the release results of mitochondria-targeted prodrug 17 in Hela cells in an experimental example of the present disclosure, wherein A is real-time viable cell imaging photos of prodrug 17 (1 μM) at various incubation times in HeLa cells and fluorescence imaging photos of Dox (1 μM) after incubation for 0.5 h; and in B, i) is a real-time cell imaging photo of HeLa cells after incubation directly with prodrug 17 (1 μM) for 3 hours; ii) is a real-time cell imaging photo of HeLa cells pretreated with the GSH inhibitor BSO (5 mM) for 24 h; iii) is a fluorescence imaging photo after pretreatment with biotin (1 mM) for 1 h and then co-incubation with prodrug 17 (1 μM) for 3 h, or iv) is a fluorescence imaging photo after pretreatment with biotin (2 mM) for 1 h and then co-incubation with prodrug 17 (1 μM) for 3 h; and v) is an imaging photo of viable cells co-incubated with prodrug 17 (1 μM) for 1 hour and then with GSH (2 mM) for another 1 hour (incubation with prodrug 17 for totally 2 hours); and

FIG. 25 is a graph showing the results of a cellular function study on prodrug 17 obtained after connecting MTP3 to Dox in an experimental example of the present disclosure, wherein A is a mitochondrial co-localization imaging photo of prodrug 17 (1 μM) in HeLa cells; B is a mitochondrial membrane potential change graph after treating HeLa cells with prodrug 17 (0-10 μM); C is a graph showing the results of the effects of prodrug 17 and the active pharmaceutical ingredient Dox at various concentrations on the cell viability of HeLa tumor cells; D is a graph showing the results of the toxicities of prodrug 17 and the active pharmaceutical ingredient Dox at various concentrations to normal CHO cells; E is the cell viabilities of HeLa cells treated by the mitochondria-targeted peptides MTP2, MTP3, MTP4 and MTPS at various concentrations; F is a graph showing the nuclear morphological change of HeLa cells observed after treating the HeLa cells with prodrug 17 (2 μM) for 24 hours and then staining the HeLa cells with Hoechst 33342; and G is a graph showing the flow cytometry results after co-incubation of HeLa cells with prodrug 17 at various concentrations for 24 h, as detected using Annexin V-PE Apoptosis Kit.

### DETAILED DESCRIPTION

The concepts of the present disclosure and the resulting technical effects are described clearly and completely below in conjunction with examples in order to fully understand the objects, features, and effects of the present disclosure. Obviously, the described examples are only some, rather than all, of the examples of the present disclosure. Based on the examples of the present disclosure, other examples obtained by a person skilled in the art without involving any inventive effort also fall within the scope of the present disclosure.

### Example 1 Preparation of Mitochondria-Targeted Polypeptide

In this example, a mitochondria-targeted polypeptide (MTP) was prepared, and the specific process was as follows:

1. Synthesis of Rink Amide Resin

Tentagel resin (0.26 mmol/g, 2 g, 1 eq) was placed in a polypeptide solid-phase synthesis tube, DCM was added to swell the resin, the tube was gently shaken for 3 minutes, and the solvent was then removed by vacuum filtration; this operation was repeated three times. DMF was added to the resin, the mixture was gently shaken for 3 minutes, and the solvent was then removed by vacuum filtration; this operation was repeated three times. Rink Amide linker (1.1232 g, 4 eq) and HATU (0.7904 g, 4 eq) were dissolved in DMF, DIEA (0.72 mL, 8 eq) was then added, the mixture was sufficiently mixed, and the solution was poured into the resin and shaken overnight. After the reaction was completed; the remaining solution was suctioned out by vacuum filtration. Washing with DMF, DCM and DMF were performed three times respectively.

2. Synthesis of Mitochondria-Targeted Polypeptide (MTP)-Resin Complex by Fmoc Solid-Phase Synthesis Process

A pentapeptide skeleton with a sequence of Fmoc-L-Nal-D-Nal-Gly-Gly-Lys(Mtt) was synthesized using the Rink Amide resin obtained in step 1 by an Fmoc-protection solid-phase synthesis process, and the resulting pentapeptide molecular skeleton was then condensed with arginine to obtain the linear polypeptide molecular chain Fmoc-Arg(Pbf)-[Arg(Pbf)]n-Arg(Pbf)-L-Nal-D-Nal-Gly-Gly-Lys(Mtt) that contained different numbers of arginine. The specific steps were as follows:

(1) The Rink Amide resin (0.52 mmol) was added to a polypeptide solid-phase synthesis tube and swelled with DMF.

(2) Deprotection: The resin was washed with a 20% piperidine/DMF (by volume) solution. A 20% piperidine/DMF solution was added, after reaction for 15 min, the solvent was removed by vacuum suction filtration. The deprotection was repeated once to completely remove the Fmoc protecting group.

(3) Washing: The remaining solution was suctioned out by vacuum filtration. The resin was washed three times separately with DMF, DCM and DMF.

(4) Amino acid coupling: Fmoc-Lys(Mtt)-OH (1.3 g, 4 eq) and HATU (0.7910 g, 4 eq) were dissolved in DMF, and DIEA (0.72 mL, 8 eq) was added and mixed until uniform. The solution was poured into the resin and reacted for 2 h. After the reaction was completed, the resin was washed three times separately with DMF, DCM and DMF. A ninhydrin detection assay was carried out. If positive, a blocking operation step with acetic anhydride was required.

(5) Blocking (if negative, this step was not necessary): Acetic anhydride (0.98 mL, 40 eq) and DIEA (2.72 mL, 60 eq) were mixed in DCM until uniform. The solution was poured into the resin, reacted for 2 h, and then sufficiently washed with DMF, DCM and DMF for the next step of reaction.

(6) The cyclic operations of coupling, washing, deprotection, washing, and amino acid coupling were sequentially carried out until the last amino acid was coupled to the resin.

(7) After the coupling of the last amino acid was completed, acetylation was carried out. Acetic anhydride (0.98 mL, 40 eq) and DIEA (2.72 mL, 60 eq) were mixed in DCM until uniform. The washing, deprotection, and washing operations were repeated. The solution was poured into the resin and reacted for 3 h.

3. Cleavage of Polypeptide

The resulting resin in the mitochondria-targeted polypeptide (MTP) synthesized by the Fmoc solid-phase synthesis process in step 2 was washed three times separately with DMF, DCM and DMF, the resin was then washed three times with methanol, and the solvent was drained by vacuum filtration. An acidic shearing reagent (92.5% of TFA, 2.5% of water, 2.5% of TIPS, and 2.5% of 1,3-dimethoxybenzene, by v/v) was added, and a reaction was carried out for 3 h. The cleaved mixture was filtered directly into 50 mL of pre-chilled diethyl ether and maintained at −20° C. overnight. After centrifugation, the supernatant was discarded. The precipitate was re-suspended with cold diethyl ether and centrifuged, and the supernatant was discarded; this operation was repeated three times to obtain a crude polypeptide product. After the crude product was identified as the target product by LC-MS characterization, the crude product was purified by HPLC preparation and freeze-dried in vacuo to obtain high-purity target polypeptide MTP.

### Example 2 Preparation of Mitochondria-Targeted Polypeptides with Different Structures

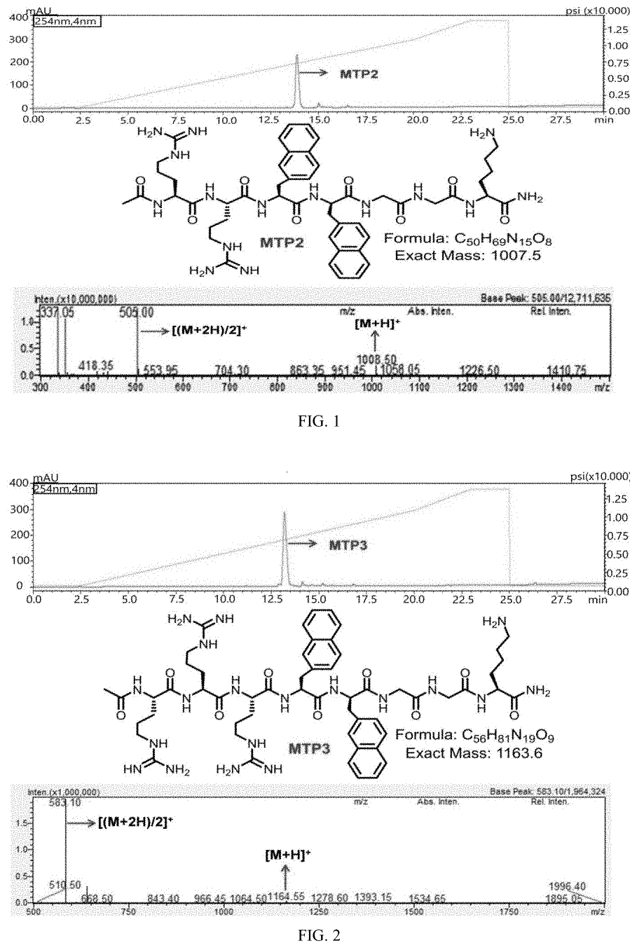

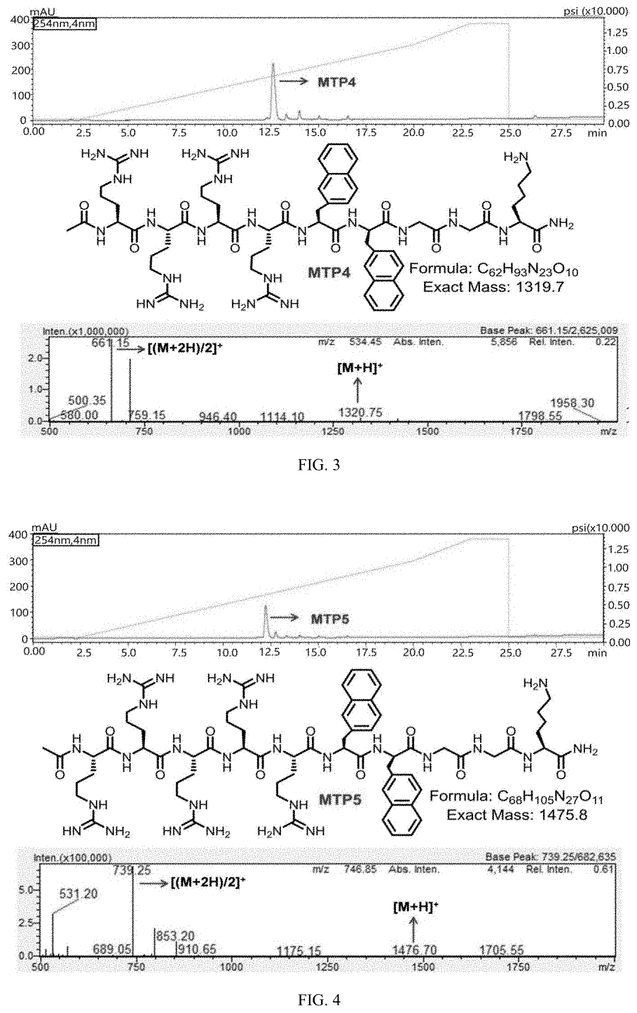

In this example, mitochondria-targeted polypeptides with different structures were prepared, wherein the structures thereof were as shown in Table 1, and the preparation method was as shown in Example 1. By the design of sequences 1 (intermediate pentapeptide), 2 (MTP2), 3 (MTP3), 4 (MTP4) and 5 (MTPS) and by changing the arginine number in the sequences, the effects of arginine number on mitochondrial localization were studied. The mitochondria-targeted peptides with different structures were characterized by LC-MS (the results were as shown in FIGS. 1-4).

TABLE 1

| Name | Sequence |
|---|---|
| 1. Intermediate | Ac-L-Nal-D-Nal-Gly-Gly-Lys |
| pentapeptide | |
| 2. MTP2 | Ac-Arg-Arg-L-Nal-D-Nal-Gly-Gly-Lys |
| 3. MTP3 | Ac-Arg-Arg-Arg-L-Nal-D-Nal-Gly-Gly-Lys |
| 4. MTP4 | Ac-Arg-Arg-Arg-Arg-L-Nal-D-Nal-Gly-Gly-Lys |
| 5. MTP5 | Ac-Arg-Arg-Arg-Arg-Arg-L-Nal-D-Nal-Gly-Gly-Lys |

The results of the characterization experiments were as shown in FIGS. 1-4, wherein FIGS. 1-4 respectively showed the LC-MS characterization results of the peptides MTP2, MTP3, MTP4, and MTPS. It could be seen from the figures that the methods of the present disclosure could prepare pure MTPs.

### Example 3 Synthesis of Mitochondria-Targeted Fluorescent Probes

Mitochondria-targeted fluorescent probes were synthesized from the mitochondria-targeted polypeptides and used as carriers for the delivery of different types of fluorescent groups.

1. Synthesis of Mitochondria-Targeted Fluorescent Probes 8-12, with the Synthesis Steps as Follows:

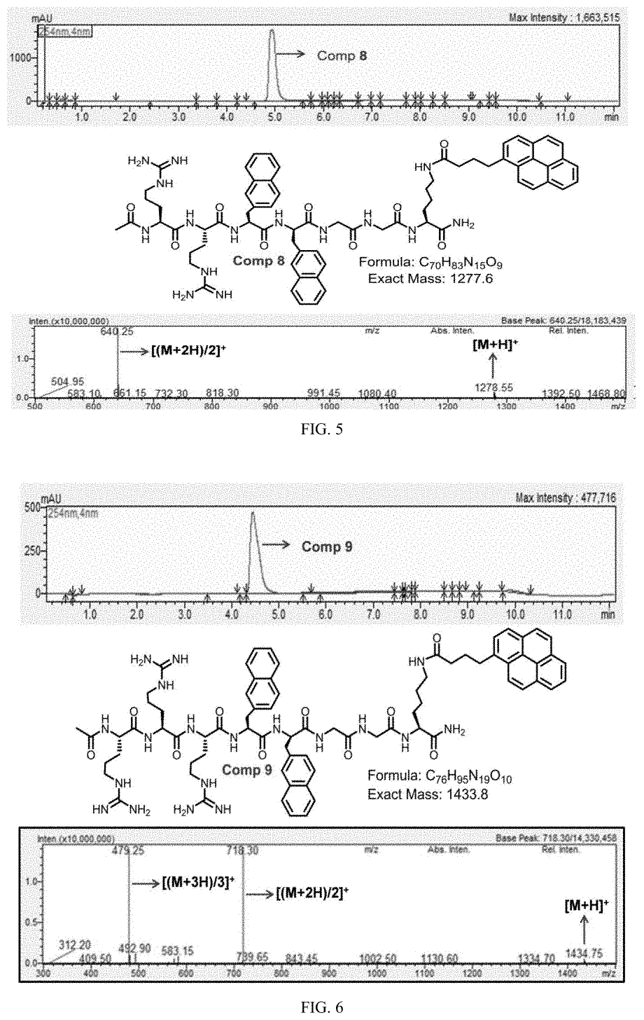

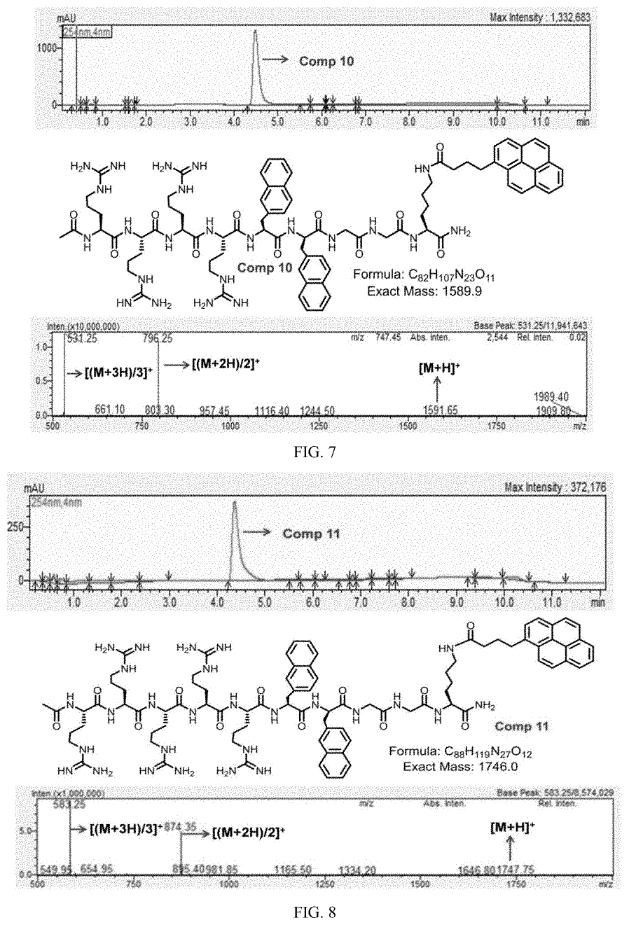

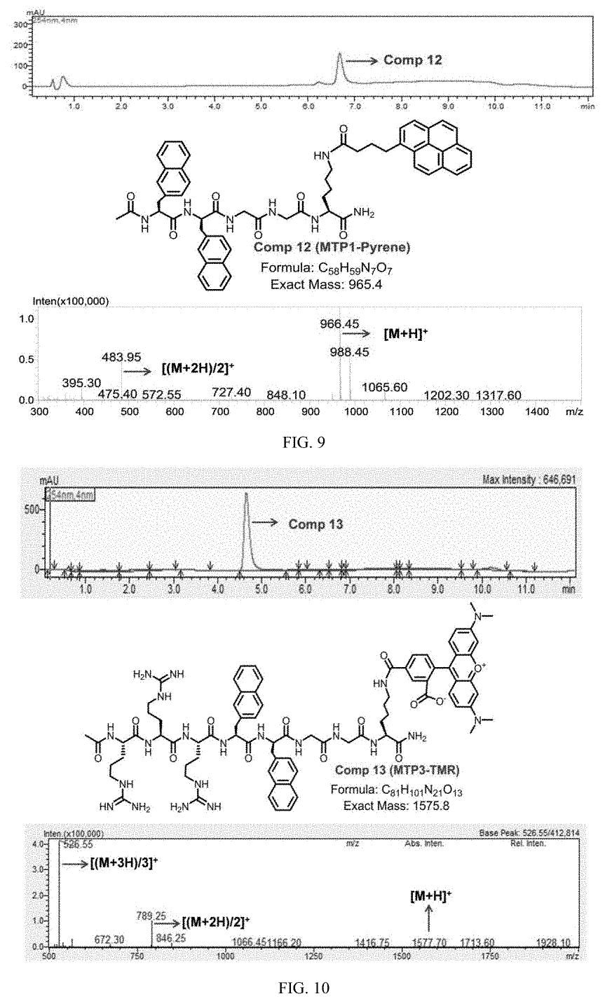

The synthesized mitochondria-targeted polypeptides 1, MTP2, MTP3, MTP4 and MTPS (6×10⁻³ mmol, 1 eq) were respectively dissolved with DMF. 1-Pyrenebutyric acid (18×10⁻³ mmol, 3 eq) was dissolved in DMF and pre-activated with HOBt (18×10⁻³ mmol, 3 eq), HBTU (18×10⁻³ mmol, 3 eq) and TEA (36×10⁻³ mmol, 6 eq). The pre-activated solution was mixed with the mitochondria-targeted polypeptide solutions and then stirred overnight at room temperature, and the resulting crude samples were purified by preparative high performance liquid chromatography. Characterization was carried out by LC-MS analysis, and the characterization results were as shown in FIGS. 5-9.

The experimental results were as shown in FIGS. 5-9. As could be seen from the figures, FIGS. 5-9 respectively showed the LC-MS characterization results of mitochondria-targeted fluorescent probes 8-12 obtained after MTP2, MTP3, MTP4, MTPS and the intermediate pentapeptide were respectively connected to a pyrene-based fluorophore, indicating that the method of the present disclosure could prepare pure mitochondria-targeted fluorescent probe molecules.

2. Synthesis of Mitochondria-Targeted Fluorescent Probe 13, with the Synthesis Steps as Follows:

The synthesized mitochondria-targeted polypeptide MTP3 (4 mg, 1 eq), TMR-NHS active ester (2 eq) and TEA (4 eq) were mixed in dry DMF until uniform, the mixture was stirred and reacted at room temperature for 24 h, and the reaction product was purified by preparative high performance liquid chromatography. Characterization was carried out by LC-MS analysis, and the characterization results were as shown in FIG. 10. It could be seen from the figure that the present disclosure prepared a pure MTP3-TMR fluorescent probe.

3. Synthesis of Mitochondria-Targeted Fluorescent Probe 14, with the Synthesis Steps as Follows:

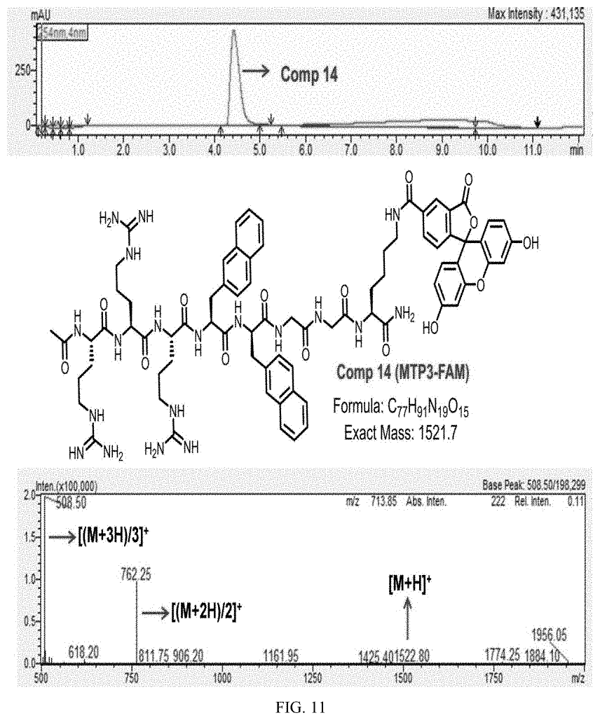

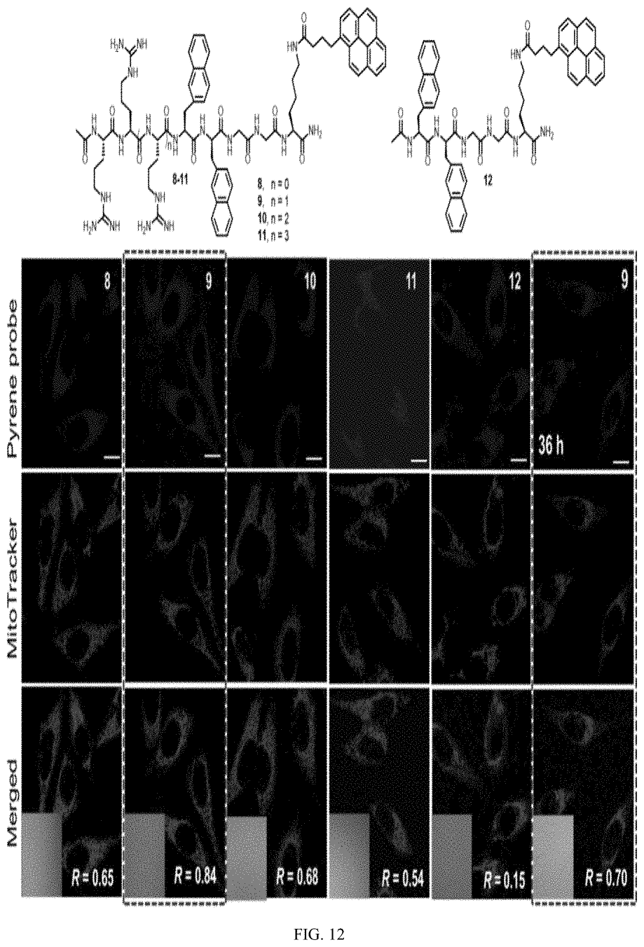

The synthesized mitochondria-targeted polypeptide MTP3 (4 mg, 1 eq), 5-FAM-NHS active ester (2 eq) and TEA (4 eq) were mixed in dry DMF until uniform, the mixture was stirred and reacted at room temperature for 24 h, and the reaction product was purified by preparative high performance liquid chromatography. Characterization was carried out by LC-MS analysis, and the characterization results were as shown in FIG. 11. It could be seen from the figure that the present disclosure prepared a pure MTP3-FAM fluorescent probe.

### Test Examples

1. Study of the Effect of the Arginine Number on the Mitochondrial Localization of Synthesized Mitochondria-Targeted Fluorescent Probes

The synthesized mitochondria-targeted fluorescent probes were analyzed by confocal imaging to detect the effect of the arginine number on the mitochondrial localization thereof.

Experimental method: Solid powders of mitochondria-targeted fluorescent probes 8-12 synthesized in Example 3 were dissolved in DMSO to prepare 2 mM test mother solutions, and HeLa cells were cultured in a culture medium (DMEM: FBS: penicillin-streptomycin dual antibody=9:1:0.1). The cells were inoculated in an 8-well imaging dish and cultured in a cell incubator containing 5% CO₂ at 37° C. until the cell density reached 60%. After the culture medium was removed, the cells were incubated with the target compound for a specified time. The culture medium was then removed, and wash with PBS was conducted three times. Thereafter, 50 nM commercial mitochondrial probe MitoTracker Deep Red (50 nM) was further added to the culture dish for 15 minutes of continuous incubation. After the culture medium was removed, wash with PBS was conducted three times. Imaging on confocal microscope was then carried out. Blue channel tracker 1 was set, with the excitation wavelength being 405 nm and the emission band being 420-490 nm, and this channel was used to receive fluorescence emitted by the mitochondria-targeted peptide. Red channel tracker 2 was set, with the excitation wavelength being 640 nm and the emission band being 655-755 nm, and this channel was used to receive fluorescence emitted by MitoTracker Deep Red, a commercial mitochondrial dye. In addition, in a cell imaging experiment, green channel tracker 3 was set, with the excitation wavelength being 560 nm and the emission band being 580-620 nm, and this channel was used to receive fluorescence emitted by compound 13. Green channel tracker 4 was set, with the excitation wavelength being 488 nm and the emission band being 500-600 nm, and this channel was used to receive fluorescence emitted by compound 14.

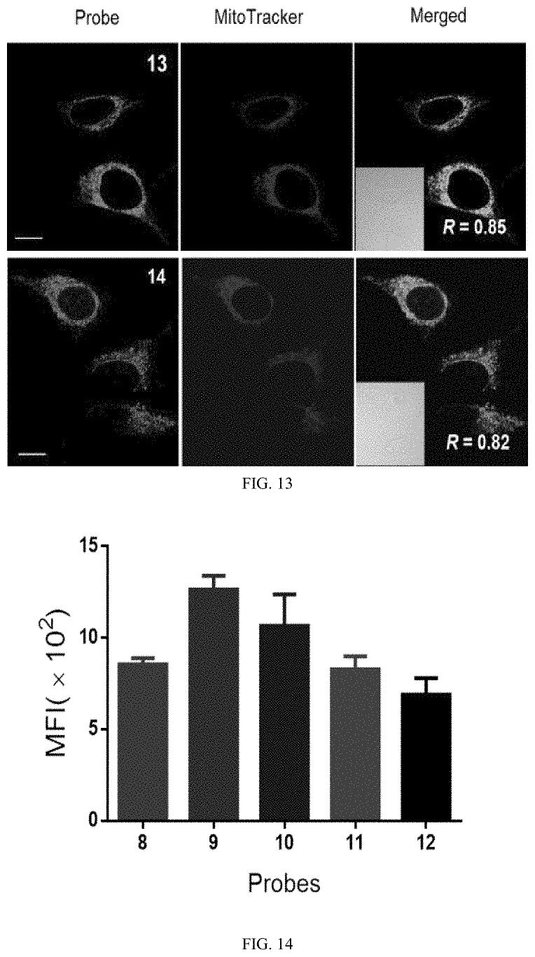

The experimental results were shown in FIGS. 12 and 13. It could be seen from FIG. 12 that MTP3 had relatively high mitochondrial co-localization ability, and the co-localization coefficient of MTP3 with the commercial mitochondrial probe was 0.84. The co-localization coefficient of MTP2 with the commercial mitochondrial dye was 0.65, the co-localization coefficient of MTP4 with the commercial mitochondrial dye was 0.68, and the co-localization coefficient of MTPS with the commercial mitochondrial dye was 0.54. Whereas the co-localization coefficient of the control probe without any arginine residue (compound 12) with the commercial mitochondrial dye was 0.24. It could be seen from FIG. 13 that the prepared mitochondria-targeted polypeptide could be effectively localized to mitochondria, especially the MTP3 probe, which had a co-localization coefficient of still up to 0.70 with the commercial fluorescent probe after co-incubation with cells for up to 36 h, indicating that this probe could be used for long-term tracing imaging of mitochondrial targeting. In addition, after modification with various fluorescent groups, the mitochondria-targeted polypeptide MTP3 prepared by the present application still had similar mitochondrial targeting properties. The co-localization coefficient of MTP3-TMR (compound 13) with the commercial mitochondrial dye was 0.85, and the co-localization coefficient of MTP3-FAM (compound 14) with the commercial mitochondrial dye was 0.82. Therefore, the mitochondrial localization ability of the mitochondria-targeted polypeptide provided by the present disclosure was independent of fluorescent groups, and it was expected to be applied to targeted delivery of an exogenous bioactive drug to mitochondria. These results indicated that the mitochondria-targeted polypeptide provided by the present disclosure had the advantages of easy modification and stable localization to mitochondria.

2. Study of the Effect of the Arginine Number on the Cell Uptake of the Synthesized Mitochondria-Targeted Fluorescent Probe

Experimental steps: Cells were inoculated in an 8-well imaging dish and cultured overnight in a 5% CO₂ environment at 37° C.; after the culture medium was removed, the cells were co-incubated with cell culture fluids containing mitochondria-targeted fluorescent probes 8-12 (2 μM), which had different arginine numbers, for 2 h; after the culture medium was removed, the cells were washed with PBS; and then, the cell uptake was observed by confocal fluorescence microscopy and subsequently counted.

The experimental results were as shown in FIG. 14. It could be seen from the figure that the mitochondria-targeted fluorescent probes with different arginine numbers could all be effectively taken up by cells, and compound 9 had stronger cell uptake ability.

3. Synthesis of Mitochondria-Targeted Prodrug (Compound 17)

(1) Synthesis of Tumor-Targeted and Mitochondria-Targeted Compound 16

The structural formula of the compound 16 was as shown below:

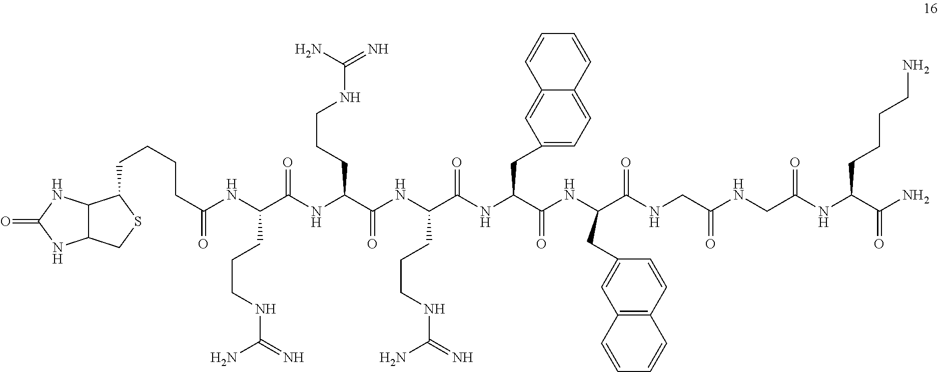 [SMILES: N=C(N)NCCC[C@H](NC(=O)CCCC[C@@H]1SCC2NC(=O)NC21)C(=O)N[C@@H](CCCNC(=N)N)C(=O)N[C@@H](CCCNC(=N)N)C(=O)N[C@@H](Cc1ccc2ccccc2c1)C(=O)N[C@H](Cc1ccc2ccccc2c1)C(=O)NCC(=O)NCC(=O)N[C@@H](CCCCN)C(N)=O]

The synthesis steps were as follows:

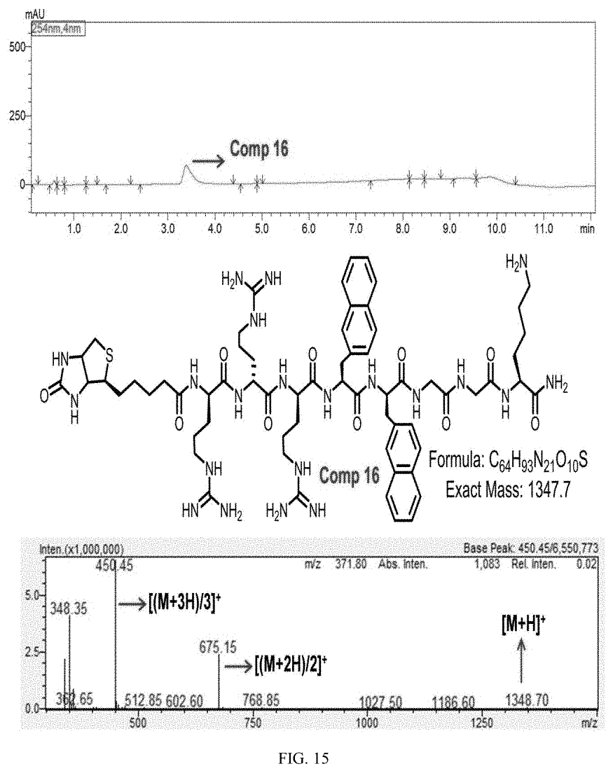

The MTP3-resin complex (1 eq) obtained in Example 1, which had not been cleaved from the resin, was added to a solution of biotin (D-biotin, also known as vitamin H) (3 eq), the coupling agent HATU (3 eq) and DIEA (6 eq) in DMF. The reaction mixture was shaken at room temperature for 6 hours. The resin was washed three times separately with DMF, DCM, and DMF and then washed three times with methanol, and the solvent was drained by vacuum filtration. The resin was then cleaved using the method provided in Example 1. The crude product was purified by preparative high performance liquid chromatography to obtain compound 16, which was characterized by LC-MS. The results were as shown in FIG. 15. It could be seen from the figure that the method of the present disclosure successfully prepared pure compound 16.

(2) Synthesis of GSH-Responsive Disulfide Linker Compound 19

The structural formula of compound 19 was as shown below:

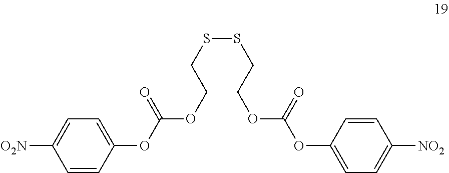 [SMILES: O=C(OCCSSCCOC(=O)Oc1ccc([N+](=O)[O-])cc1)Oc1ccc([N+](=O)[O-])cc1]

The specific synthesis steps were as follows:

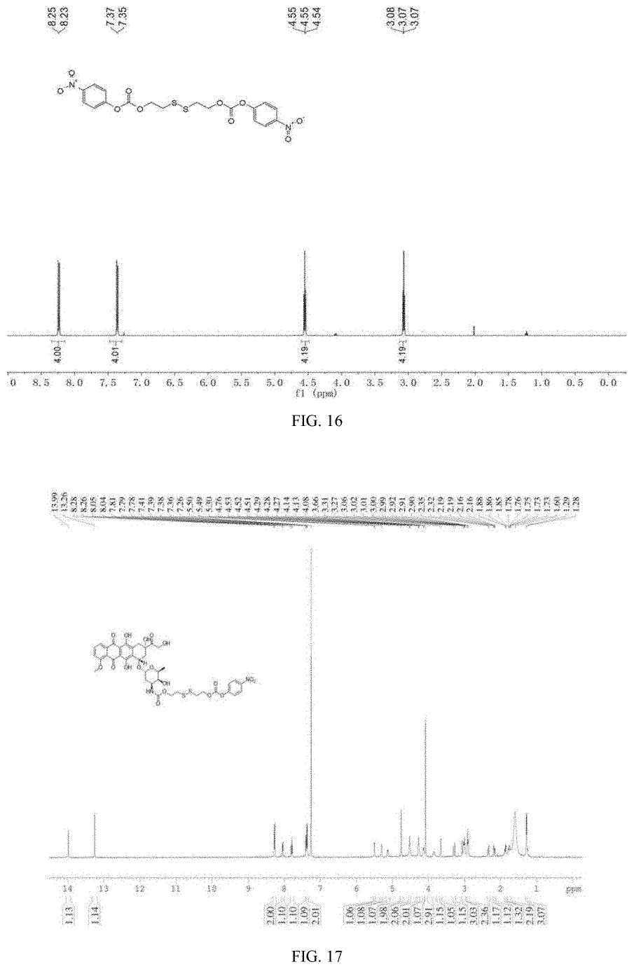

p-Nitrophenyl chloroformate (13.0 mmol, 2.5 eq) and DIEA (13.0 mmol, 2.5 eq) were dissolved with dry DCM. A DCM solution containing 2-hydroxyethyl disulfide (5.2 mmol, 1.0 eq) was added at 0° C. The reaction mixture was stirred at room temperature for 8 hours. After the solvent was removed, the resulting mixture was redissolved in 50 mL of ethyl acetate and washed sequentially with saturated brine and water. The organic layer was dried over anhydrous sodium sulfate, filtered and evaporated under reduced pressure. The crude product was purified by silica gel flash column chromatography to obtain compound 19 (white solid, 1.72 g, 68%). Characterization by ¹H NMR analysis was carried out. ¹H NMR (500 MHz, CDCl₃), δ: 8.24 (d, J=10 Hz, 4H), 7.36 (d, J=10 Hz, 4H), 4.55 (t, J=4 Hz, 4H), 3.08 (t, J=4 Hz, 4H) ppm. The results were as shown in FIG. 16. It could be seen from the figure that the method of the present disclosure successfully prepared compound 19.

(3) Synthesis of Compound 20 (Dox-SS-PNCC)

The structural formula of compound 20 was as shown below:

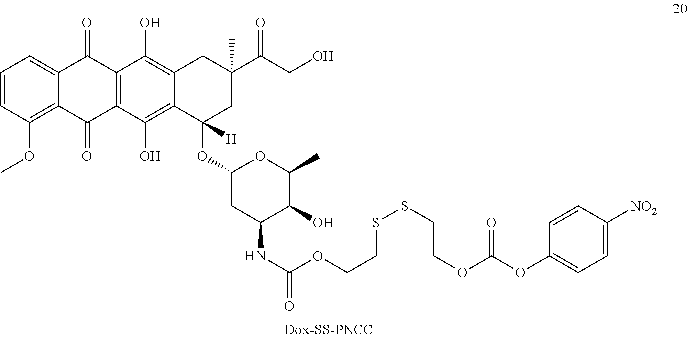 [SMILES: COc1cccc2c1C(=O)c1c(O)c3c(c(O)c1C2=O)C[C@@](C)(C(=O)CO)C[C@@H]3O[C@H]1C[C@H](NC(=O)OCCSSCCOC(=O)Oc2ccc([N+](=O)[O-])cc2)[C@H](O)[C@H](C)O1]

The specific synthesis steps were as follows:

A DMF solution of DOX hydrochloride (91 mg, 0.16 mmol) and TEA (64 μL, 0.47 mmol) was slowly added to a stirred DMF solution of compound 19 (100 mg, 0.19 mmol) at 0° C., and a reaction was carried out with stirring at room temperature and monitored by thin layer chromatography. After the reaction was completed, 20 mL of water was added to the reaction mixture, and the product was extracted with ethyl acetate. The combined organic phases were distilled under reduced pressure to remove the solvent, and the remaining red residue was purified by silica gel column chromatography to obtain compound 20 (red solid, 0.1 g, 72%), which was characterized by ¹H NMR and ¹³C NMR. ¹H NMR (500 MHz, CDCl₃) δ: 13.98 (s, 1H), 13.26 (s, 1H), 8.27 (d, J=9.1 Hz, 2H), 8.04 (d, J=6.1 Hz, 1H), 7.81-7.78 (m, 1H), 7.40 (d, J=8.5 Hz, 1H), 7.37 (d, J=9.1 Hz, 1H), 5.50-5.49 (m, 1H), 5.30 (br, 1H), 5.14-5.12 (m, 1H), 4.76 (s, 2H), 4.52 (t, J=6.3 Hz, 2H), 4.28 (t, J=6.1 Hz, 2H), 4.14-4.13 (m, 1H), 4.06 (s, 3H), 3.85 (br, 1H), 3.66 (br, 1H), 3.31-3.27 (m, 1H), 3.06-2.99 (m, 3H), 2.92-2.90 (m, 2H), 2.35-2.32 (m, 1H), 2.19-2.16 (m, 1H), 1.89-1.85 (m, 1H), 1.79-1.73 (m, 2H), 1.28 (d, J=6.5 Hz, 3H), ¹³C NMR (126 MHz, CDCl₃) δ: 213.77, 186.80, 186.42, 162.50, 160.87, 156.07, 155.42, 155.25, 152.16, 145.31, 135.67, 135.22, 133.59, 133.50, 125.19, 121.68, 111.33, 111.16, 100.66, 76.47, 69.44, 69.20, 67.35, 66.72, 65.39, 62.35, 56.53, 46.97, 37.62, 36.42, 35.50, 33.74, 31.35, 29.95, 29.56, 16.77 ppm. ESI-MS: [M-1]-: calcld 887.2; found 887.2. The characterization results were as shown in FIG. 17. It could be seen from the figure that the method of the present disclosure successfully prepared compound 20.

(4) Synthesis of Mitochondria-Targeted Prodrug Compound 17

The structural formula of prodrug 17 (Bio-MTP3-SS-Dox) was as shown below:

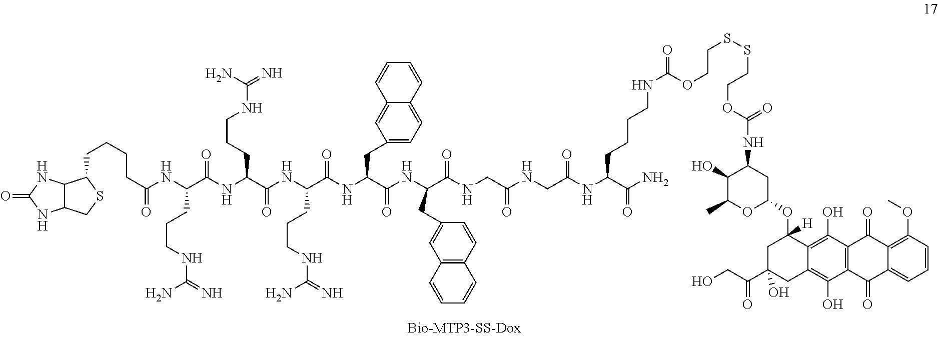 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSCCOC(=O)NCCCC[C@H](NC(=O)CNC(=O)CNC(=O)[C@@H](Cc2ccc3ccccc3c2)NC(=O)[C@H](Cc2ccc3ccccc3c2)NC(=O)[C@H](CCCNC(=N)N)NC(=O)[C@H](CCCNC(=N)N)NC(=O)[C@H](CCCNC(=N)N)NC(=O)CCCC[C@@H]2SCC3NC(=O)NC32)C(N)=O)[C@H](O)[C@H](C)O1]

The synthesis steps were as follows:

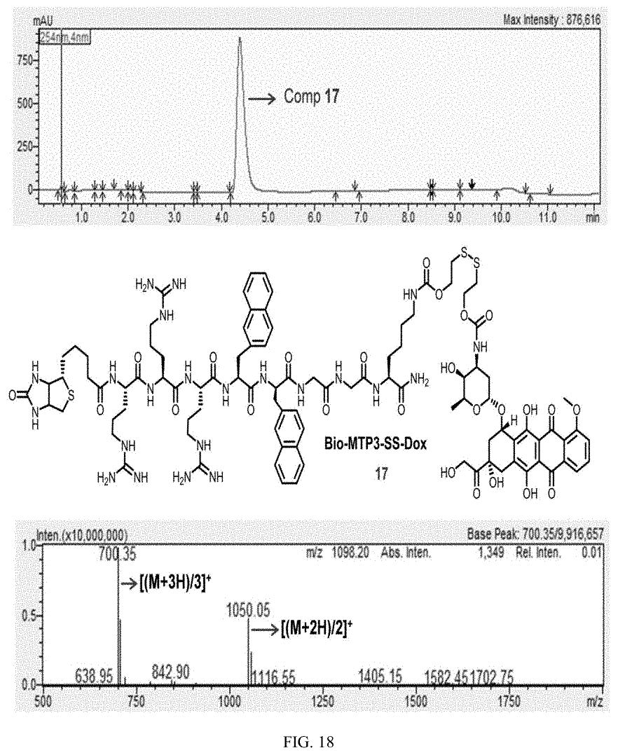

Compound 16 (1.0 eq), TEA (2 eq) and compound 20 (1.2 eq) were dissolved in dry DMF. After shaking at room temperature for 24 h, the resulting mixture was purified by preparative high performance liquid chromatography to obtain compound 17(red solid), which was characterized by LC-MS. The results were as shown in FIG. 18. It could be seen from the figure that the method of the present disclosure successfully prepared mitochondria-targeted prodrug compound 17.

4. Drug Test of Mitochondria-Targeted Prodrug (Compound 17)

Mitochondria-targeted prodrug compound 17 prepared in Test Example 3 was subjected to an in vitro test, specifically as follows:

(1) Drug Release Test of Mitochondria-Targeted Prodrug 17 in Phosphate Buffered Saline Solution

Prodrug 17 (5 mM stock solution in DMSO) was diluted with PBS to form a solution at an indicated concentration and then incubated at 37° C. with a glutathione (GSH) solution at a specified concentration (0-12 mM GSH for concentration-dependent study and 10 mM GSH for time-dependent study), and the fluorescence of the treated sample was monitored periodically at specific time points. The change in the fluorescence intensity of the sample reflected the drug release behavior of prodrug 17 under GSH activation. In addition, a chromatogram was detected by high performance liquid chromatography after co-incubation of prodrug 17 (20 μM) with GSH (10 mM) in a PBS solution at 37° C. for 6 h, and a mass spectrum of the reaction solution was detected by ESI-MS.

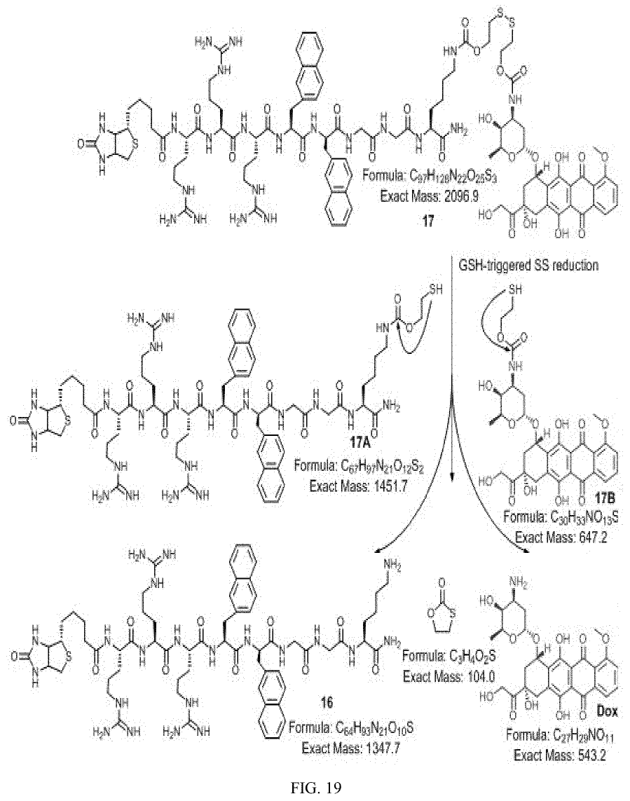

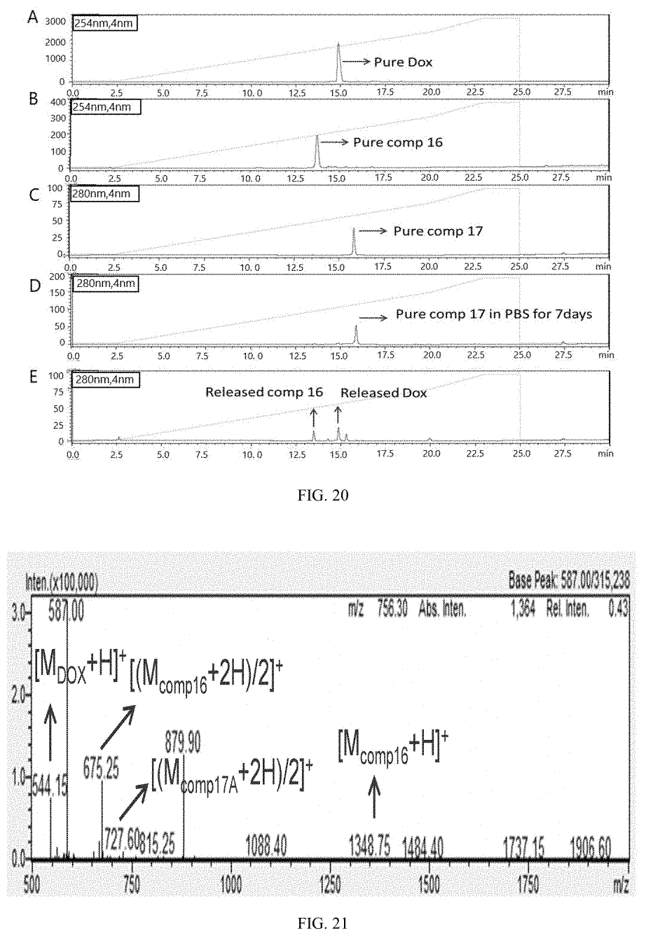

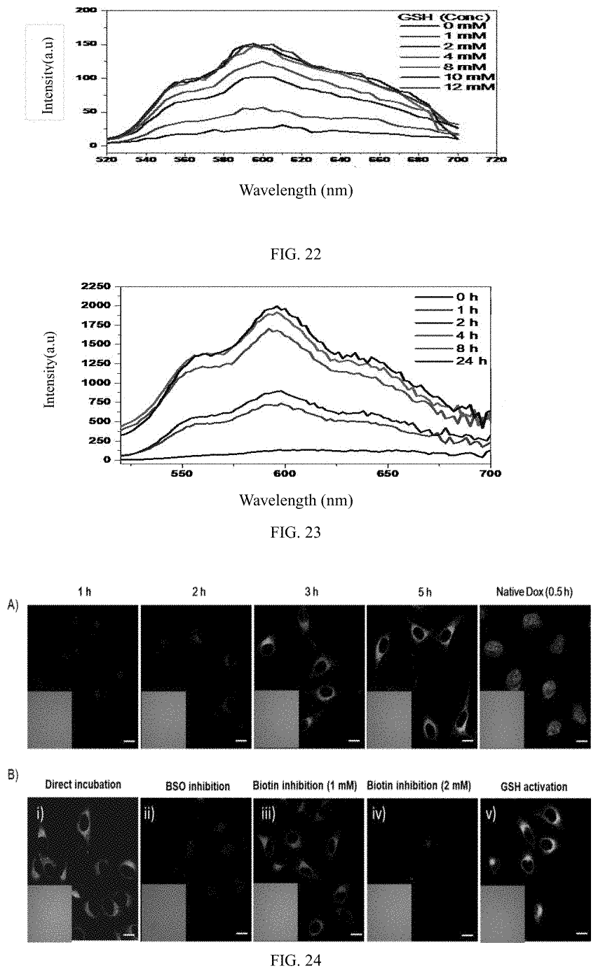

The experimental results were as shown in FIGS. 19-23, wherein FIG. 19 was a schematic diagram showing the GSH-responsive release mechanism of mitochondria-targeted prodrug 17 in a PBS solution in vitro; FIG. 20 was a graph showing the GSH-responsive drug release results of mitochondria-targeted prodrug 17, as detected by HPLC; FIG. 21 was a graph showing the release results of mitochondria-targeted prodrug 17, as detected by ESI-MS; FIG. 22 was a graph showing the GSH concentration-dependent results for the drug release of mitochondria-targeted prodrug 17; and FIG. 23 was a graph showing the responsive release results of mitochondria-targeted prodrug 17 in a PBS solution in vitro over time. It could be seen from the figures that prodrug 17 provided by the present disclosure was stable, and no obvious compound degradation was found even after placement in the PBS solution at 37° C. for 7 days; in addition, after co-incubation with GSH for 6 h, the released Dox and mitochondria-targeted peptide 16 could be clearly detected by high performance liquid chromatography and confirmed by ESI-MS. In addition, the release of the active pharmaceutical ingredient from prodrug 17 under the triggering of GSH showed obvious GSH concentration-dependent and time-dependent manner. At 37° C. and under the action of 10 mM GSH, the tested fluorescence of the sample gradually increased and reached the maximum value at about 8 hours, indicating that the drug release was relatively mild, thus avoiding acute toxicity caused by excessively quick drug release in the animal study.

(2) Drug Release Test of Mitochondria-Targeted Prodrug 17 in Cells.

Hela cells were cultured in an 8-well imaging dish and then treated with prodrug 17 (1 μM) for a certain time (1 h, 2 h, 3 h and 5 h). After the culture medium was removed, the cells were washed with PBS, a fresh culture medium was used for replacement, and the treated cells were then photographed by a fluorescence microscope. In addition, the cells were directly co-incubated with compound 17 (1 μM) for 1 h and then incubated with exogenous GSH (2 mM) for another 1 h (totally 2 h of incubation with compound 17); the cells were pretreated with the GSH inhibitor BSO (5 mM) for 24 h and then incubated with compound 17 (1 μM) for 3 h; and the cells were pre-incubated with a tumor-targeted biotin ligand at various concentrations (1 mM or 2 mM) for 1 h and then co-incubated with compound 17 for 3 h. After the culture medium was removed, the cells were washed with PBS, a fresh culture medium was used for replacement, and a fluorescence microscope was then used to take an image, which was used as the control.

The experimental results were as shown in FIG. 24, wherein A were photos of real-time viable cell imaging of prodrug 17 (1 μM) at various incubation time slots in HeLa cells and the fluorescence imaging of Dox (1 μM) after incubation for 0.5 h; and in B, i) was a real-time cell imaging photo of HeLa cells after incubation directly with prodrug 17 (1 μM) for 3 hours; ii) was a real-time cell imaging photo of HeLa cells pretreated with the GSH inhibitor BSO (5 mM) for 24 h; iii) was a fluorescence imaging photo after pretreatment with biotin (1 mM) for 1 h and then co-incubation with prodrug 17 (1 μM) for 3 h, or iv) was a fluorescence imaging photo after pretreatment with biotin (2 mM) for 1 h and then co-incubation with prodrug 17 (1 μM) for 3 h; and v) was an imaging photo of viable cells co-incubated with prodrug 17 (1 μM) for 1 hour and then with GSH (2 mM) for another 1 hour (totally 2 hours of incubation with prodrug 17). It could be seen from the figure that prodrug 17 could be slowly taken up by HeLa cells and released into the cells. In addition, the addition of exogenous GSH could accelerate the release of the drug in cells, whereas after pre-incubation with the GSH inhibitor, the cells treated under the same conditions had almost no fluorescence. It was indicated that prodrug 17 provided by the present disclosure could respond to GSH in cells and release Dox with strong fluorescence, and thus visualizing the treated cells. Considering that GSH was highly expressed in many tumor cells, prodrug 17 showed potential as a selective anti-tumor drug. Secondly, the tumor-targeted biotin ligand could also effectively inhibit the cell uptake of compound 17, thereby further improving the tumor targeting of prodrug 17. According to the present disclosure, the active pharmaceutical ingredient Dox was mainly located in the nucleus (as shown in FIG. 24A, Native Dox), whereas the modified prodrug was mainly located in mitochondria. Therefore, the mitochondria-targeted polypeptide provided by the present disclosure could not only transport the drug into cells but also reprogram the localization and distribution of the active pharmaceutical ingredient in the cells.

(3) Cellular Function Study of Mitochondria-Targeted Prodrug 17

1) Cytotoxicity Test

Cells were inoculated in a 96-well plate and cultured overnight in a cell incubator containing 5% CO₂ at 37° C. The cells were treated with prodrug 17 at various concentrations and cultured in a cell incubator containing 5% CO₂ at 37° C. for 48 h. A commercial MTT assay kit was then used to measure the cell viability. The experiment was repeated three times, and the data were analyzed by GraphPad Prism 6.0 software.

2) Determination of Mitochondrial Membrane Potential MMP

Cells were inoculated in a 384-well plate with a black transparent bottom and then cultured overnight in a cell incubator containing 5% CO₂ at 37° C., and the cells were treated with prodrug 17 at a specified concentration for 24 hours. An MMP kit (JC-10 dye, MAK-160) was then used for treatment and measurement according to the test method as provided, wherein apoptotic/damaged cells were monitored using λex=490 nm and λem=525 nm, and normal cells were monitored using λex=540 nm and λem=590 nm.

3) Apoptosis Detection

Cells were inoculated in a 35 mm culture dish. After culture overnight at 37° C., the cells were treated with prodrug 17 at a specified concentration for 24 h. The cells were then collected, rinsed with PBS, resuspended in 500 μL of 1×buffer, and quantitatively measured by a flow cytometer according to a method given by the apoptosis kit Annexin V-PE.

4) Observation of Nuclear Morphology

Cells were inoculated in an 8-well imaging culture dish and cultured overnight in a cell incubator containing 5% CO₂ at 37° C. After the culture medium was removed, the cells were incubated with prodrug 17 (2 μM) for 24 h and then stained with Hoechst 33342 (1 μM) for 15 minutes. The treated cells were rinsed with PBS and observed by imaging with a confocal microscope.

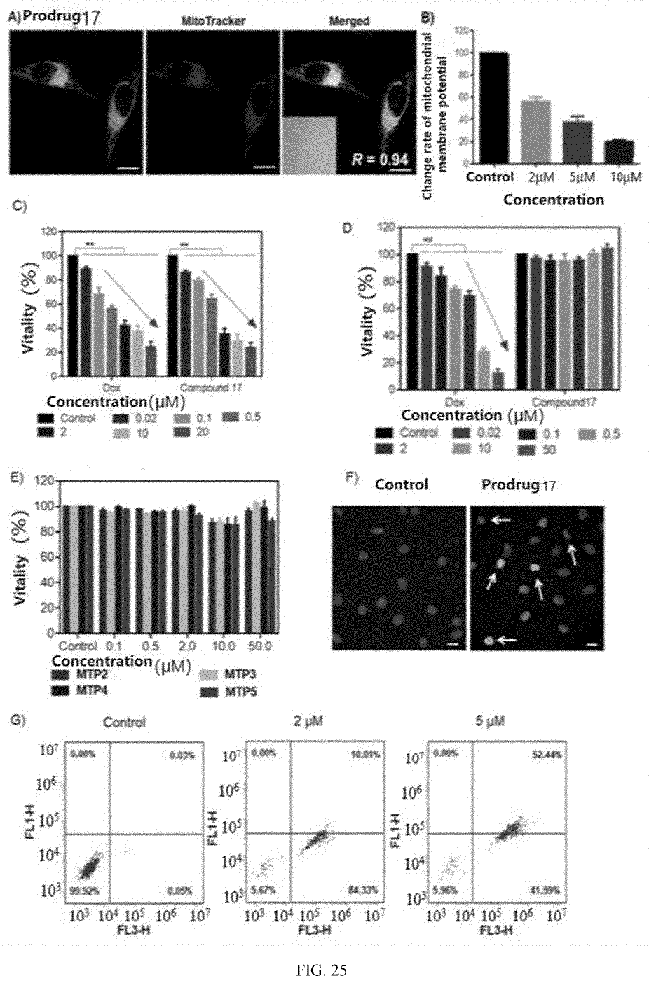

The experimental results were as shown in FIG. 25, wherein A were mitochondrial co-localization images of prodrug 17 (1 μM) in HeLa cells; B showed the mitochondrial potential changes of HeLa cells after treatment with prodrug 17 at various concentrations (0-10 μM); C showed the cell viabilities of HeLa cells upon treatment with different concentrations of Dox and prodrug 17; D was a graph showing the cell viabilities of normal CHO cells upon treatment with different concentrations of Dox and prodrug 17; E showed the cell viabilities of HeLa cells treated by various concentrations of mitochondria-targeted peptides (MTPs); F was a graph showing the nuclear morphological change of HeLa cells observed after treating HeLa cells with prodrug 17 (2 μM) for 24 hours and then staining the HeLa cells with Hoechst 33342; and G were flow cytometry results using Annexin V-PE apoptosis kit after co-incubation of HeLa cells with prodrug 17 at various concentrations for 24 h. As could be seen from the figures, prodrug 17 provided by the present disclosure exhibited excellent mitochondrial targeting properties and a strong anti-tumor activity, prodrug 17 could traceless release the active pharmaceutical ingredient Dox with strong fluorescence in mitochondria, thus inducing a significant mitochondrial membrane depolarization and leading to a remarkable reduction in mitochondrial membrane potential, thereby subsequently induce cell death through cell apoptosis. Of note, prodrug 17 exhibited a selective anti-tumor effect, with almost no toxicity to normal cells (CHO cells). In addition, the series of mitochondria-targeted peptides synthesized also showed good biocompatibility to cells, and the cells remained basically unharmed at a high concentration of up to 50 μM.

In summary, the mitochondria-targeted polypeptides prepared by the present disclosure demonstrate good mitochondrial-targeting properties and can be multi-functionally modified and transformed conveniently, and the obtained prodrug 17 can be used as an effective mitochondria-targeted therapeutic drug for tumor-targeted therapies.

The embodiments of the present disclosure have been described in detail above in conjunction with the accompanying drawings; however, the present disclosure is not limited to the above-mentioned embodiments; in addition, within the scope of knowledge possessed by those of ordinary skill in the art, various changes can also be made without departing from the spirit of the present disclosure. Furthermore, the embodiments of the present disclosure and the features in the embodiments may be combined with each other without conflict.

1. A mitochondria-targeted polypeptide, wherein the polypeptide is abbreviated as MTP, and the general structural formula of the polypeptide is as shown below in Formula I: 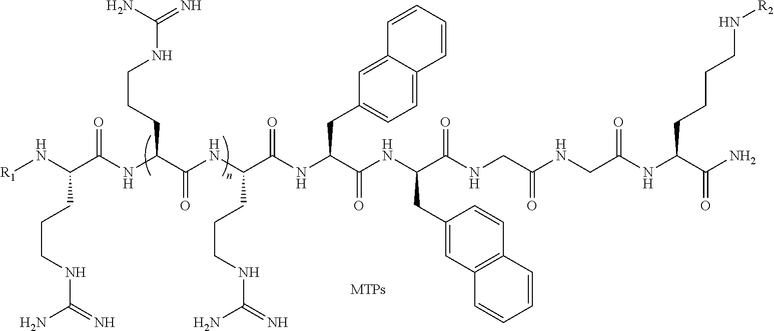
wherein n≥0, R1 is an amino protecting group or a tumor-targeting ligand, and R2 is at least one selected from the group consisting of hydrogen, fluorescent groups, and drug groups.

2. The polypeptide according to claim 1, wherein the amino protecting group is at least one selected from the group consisting of acetyl, propionyl, and butyryl.

3. The polypeptide according to claim 1, wherein the tumor-targeting ligand is at least one selected from the group consisting of folic acid, nucleic acid aptamers, RGD-targeting peptides, and biotin.

4. The polypeptide according to claim 1, wherein the fluorescent group is at least one selected from the group consisting of rhodamine fluorophore and derivatives thereof, fluorescein isothiocyanate and derivatives thereof, or pyrene-based fluorophore and derivatives thereof.

5. The polypeptide according to claim 1, wherein the drug group comprises a drug; preferably, the drug is at least one selected from the group consisting of doxorubicin, camptothecin, and derivatives thereof.

6. A method for preparing the polypeptide according to claim 1, wherein the method comprises the following steps: preparing a polypeptide chain by an Fmoc solid-phase synthesis process, and cleaving and purifying the polypeptide chain to obtain polypeptide MTP.

7. A drug carrier, comprising the polypeptide according to claim 1.

8. (canceled)

9. A cell-membrane-penetrating peptide, comprising the polypeptide according claim 1.

10. A pharmaceutical composition, comprising the polypeptide according to claim 1.

11. A method according to claim 6, wherein the amino protecting group is at least one selected from the group consisting of acetyl, propionyl, and butyryl; preferably, the tumor-targeting ligand is at least one selected from the group consisting of folic acid, nucleic acid aptamers, RGD-targeting peptides, and biotin.

12. A drug carrier according to claim 7, wherein the amino protecting group is at least one selected from the group consisting of acetyl, propionyl, and butyryl.

13. A drug carrier according to claim 7, wherein the tumor-targeting ligand is at least one selected from the group consisting of folic acid, nucleic acid aptamers, RGD-targeting peptides, and biotin.

14. A drug carrier according to claim 7, wherein the drug group comprises a drug; preferably, the drug is at least one selected from the group consisting of doxorubicin, camptothecin, and derivatives thereof.

15. A cell-membrane-penetrating peptide according to claim 9, wherein the amino protecting group is at least one selected from the group consisting of acetyl, propionyl, and butyryl.

16. A cell-membrane-penetrating peptide according to claim 9, wherein the tumor-targeting ligand is at least one selected from the group consisting of folic acid, nucleic acid aptamers, RGD-targeting peptides, and biotin.

17. A cell-membrane-penetrating peptide according to claim 9, wherein the drug group comprises a drug; preferably, the drug is at least one selected from the group consisting of doxorubicin, camptothecin, and derivatives thereof.

18. The pharmaceutical composition according to claim 10, wherein the pharmaceutical composition is mitochondria-targeted.

19. A pharmaceutical composition according to claim 10, wherein the amino protecting group is at least one selected from the group consisting of acetyl, propionyl, and butyryl.

20. A pharmaceutical composition according to claim 10, wherein the tumor-targeting ligand is at least one selected from the group consisting of folic acid, nucleic acid aptamers, RGD-targeting peptides, and biotin.

21. A pharmaceutical composition according to claim 10, wherein the drug group comprises a drug; preferably, the drug is at least one selected from the group consisting of doxorubicin, camptothecin, and derivatives thereof.
